(19) United States
(12) Patent Application Publication
(10) Pub. No.: US 2008/0057121 A1
(43) Pub. Date: Mar. 6, 2008

(54) Pharmaceutical Composition and Galenic Form Corresponding to Rapid Oral Disintegration and Method for Production of Said Composition

(76) Inventors: Olivier Broussaud, Bordeaux (FR); Jean-Luc Pougnas, Libourne (FR); Nicolas Calvet, Bordeaux (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/813,744

(22) PCT Filed: Jan. 12, 2006
(86) PCT No.: PCT/EP06/00070

§ 371 Date: Sep. 12, 2007

(30) Foreign Application Priority Data
Jan. 12, 2005 (EP) 05290077.6

(51) Int. Cl. A61K 9/16; A61K 9/20; A61P 43/00

(52) U.S. Cl. 424/465; 424/490; 424/493; 424/496; 424/497

(57) ABSTRACT

The invention is based on a known method for the cold rolling of metallic rolled stock (4), which for plastic deformation enters a roll gap (3) formed between oppositely rotating rolls (1, 2) on a run-in side and leaves the roll gap on a run-out side, deformation heat that is produced being removed by supplying an industrial gas which is at a lower temperature than the rolled stock. In order on this basis to provide a cold rolling method with which, on the one hand, corrosion of the surfaces of the rolled stock and the surfaces of the rolls in the region of the roll .gap is effectively prevented and with which, on the other hand, condensation of moisture on the rolls and a reduction of the oxygen content of the ambient air of relevance to health as a result of the supply of large amounts of inert gas are avoided, it is proposed according to the invention that a measured value for the surface temperature of at least one of the rolls is determined, and that the supply of industrial gas is set on the basis of the measured value.

The present invention relates to a pulverulent pharmaceutical composition which can be used for constituting, after compression, a galenic form giving rapid oral disintegration, to such a galenic form and to a method for producing said pharmaceutical composition.

The development of pharmaceutical compositions intended to constitute solid galenic forms which, when they are administered orally to patients who have difficulties in swallowing, such as children, elderly individuals, or individuals who are mentally deficient or who are not medically cooperative, are able to crumble (i.e. to disaggregate or to disintegrate) rapidly without requiring water, is nowadays arousing increasing interest.

The expression “solid galenic form giving rapid oral disintegration” is intended to mean, in a known manner, as defined by the European Pharmacopoeia, a non-film-coated (i.e. uncoated) tablet which, when it is placed in the mouth, disintegrates in less than three minutes before being swallowed. The time required for this disintegration is commonly called rate of hydration of the tablet.

Various solid galenic forms giving rapid oral disintegration are widely used today. They are essentially freeze drying tablets, oral effervescent tablets or else tablets with a high content of disintegration agents for obtaining immediate disintegration after being placed in the mouth.

Freeze drying tablets represent the compositions giving rapid disintegration that are most commonly used, in particular due to the fact that they make it possible to ensure flash disintegration approximately 10 seconds after they have been orally administered, because of their hygroscopicity and their high porosity. However, these freeze drying tablets have the drawbacks of being very fragile when handled due to their high friability, of being extremely sensitive to atmospheric moisture and of involving a complex technology and method of production and, consequently, high production costs. As a result, this technology is generally reserved for active ingredients that are expensive and present in the pharmaceutical composition at a low dose.

Oral effervescent tablets consist of a pharmaceutical composition comprising an effervescent couple, which starts to act as soon as it comes into contact with saliva, thus ensuring complete disintegration of the tablet. However, these effervescent tablets must necessarily be small in size in order to guarantee a disintegration in the mouth of less than 3 minutes, to limit the amount of gas emitted and to limit the unpleasant taste in the mouth that characterizes them. Another drawback of these effervescent tablets is that they are very sensitive to moisture, which implies the use of a very specific production process, typically in a controlled atmosphere.

Tablets with a high content of disintegration agents are, for example, described in patent document US-A-5 464 632. This document discloses a pharmaceutical composition which in particular comprises, as disintegration agents combined with swelling agents, a high mass fraction of carboxymethyl-cellulose and of a polyvinylpyrrolidone which is crosslinked and insoluble. This composition is obtained by mixing an active ingredient, used in the form of microcrystals or of microgranules, with a mixture of excipients subjected beforehand to granulation.

This high mass fraction of disintegration agents in the compositions according to this document has the drawback of generating a chalky taste and an undesirable feeling of dryness as soon as these tablets incorporating them are placed in the mouth.

Most of the excipients used in the compositions of tablets giving rapid disintegration are in general very soluble in an aqueous medium or else have a high swelling capacity, typically in the case of the use of disintegration agents.

In order to improve the way the tablets feel in the mouth, compositions containing predominantly excipients that are highly water-soluble, such as polyols, mannitol, xylitol or sorbitol, have in particular been developed, the high solubility of these excipients promoting rapid disintegration of the tablets as soon as they come into contact with the saliva. However, the use of these excipients, such as polyols, has the drawback of increasing the risks of sticking during the compression step and of not reducing the sensitivity of the tablets to ambient moisture.

Patent document WO-A-03/086361 recommends the use of wet granulation for obtaining a pharmaceutical composition intended to form a tablet giving rapid oral disintegration. In the examples of said document, this granulation is carried out by spraying an aqueous solution of sodium lauryl sulfate onto a pulverulent mixture comprising an active ingredient dispersed in excipients.

A major drawback of the granulation which is used in said document lies in the densifying of the powder particles that it involves, which is detrimental to the penetration of the saliva into the heart of the granule obtained even with the addition of surfactant, which goes against a rapid disintegration of the tablets.

In a known manner, the development of tablets giving rapid oral disintegration must take into account a set of physical parameters which are generally contradictory, including, for the tablets obtained:
a low friability, and preferably less than 1%, otherwise the handling of the tablets can become problematic, both during the production of said tablets (risk of the tablets being damaged during the packaging or “blister” step, in particular) and when the final user intervenes (risk of crushing of the tablets during extraction from the “blister” and of crumbling of the tablets in the user's hand),
a hardness or breaking strength which is, on the contrary, relatively high, and is typically at least 15 Newtons, and
a rate of hydration in the mouth which is less than three minutes, this rate generally being penalized if the tablets are too hard.

An objective of the present invention is to overcome the abovementioned drawbacks, and this objective is achieved in that the applicant has just discovered, surprisingly, that the spraying of an aqueous colloidal solution comprising a self-emulsifying system including a combination of at least one hydrophilic film-forming compound, at least one lubricating amphiphilic compound and at least one humectant amphiphilic compound, onto solid particles based on a mixture of excipients comprising at least one diluent, in such a way that the (sprayable colloidal solution/particles intended to receive this colloidal solution) mass ratio is less than or equal to 10%, makes it possible to obtain, after drying, a pulverulent composition which consists of said solid particles coated with a prehydrating film based on said colloidal solution and which can be used for constituting, after compression, a tablet having a rate of hydration (i.e. of disintegration in the mouth) of clearly less than 60 seconds, and even less than 30 seconds.

As aqueous colloidal solution that can be used for constituting said film, a solution or suspension of oil-in-water type or else water-in-oil type (for example of microemulsion type) comprising, firstly, an aqueous phase and, secondly, said self-emulsifying system is used.

The term “self-emulsifying system” is intended to mean, in a known manner, a system which allows the emulsion to form and to reform spontaneously without mechanical agitation or the introduction of thermal energy. In this manner, said colloidal solution is capable of reforming spontaneously when said film covering said solid particles is brought into contact with saliva.

According to the invention, it will be noted that this spraying does not in any way constitute granulation (such as the wet granulations known in the prior art), due to the fact that the particles coated with the prehydrating film according to the invention are free (i.e. do not adhere to one another) and have a particle size and an apparent density that are practically unchanged, due to the relatively low above-mentioned mass ratio which is reflected by the obtaining, at the surface, of said film of reduced thickness.

In fact, the granules obtained by granulation techniques are characterized in a known manner by an agglomerate of particles bonded to one another, which is covered with a relatively thick coating layer, unlike the thin film obtained according to the invention.

In other words, a pulverulent pharmaceutical composition according to the invention, which can be used for constituting, after compression, a galenic form giving rapid oral disintegration and which comprises said solid particles, is such that the latter are coated with said prehydrating film, such that said particles thus coated are free from one another in said composition (i.e. these particles are not bonded to one another, due to the fact that the composition is devoid of any binder).

In addition to this accelerated oral disintegration, it will be noted that the tablets according to the invention advantageously have:
a hardness or breaking strength, measured according to the European Pharmacopoeia, which is greater than 15 Newtons, and preferably greater than 20 Newtons, and
a friability, also measured according to the European Pharmacopoeia, which is less than 1%, thereby making it possible to overcome the abovementioned drawback relating to the handling of the tablets both during their production and during the intervention of the final user.

This pharmaceutical composition according to the invention can also comprise at least one active ingredient in the dispersed state in said mixture of excipients, it being possible for this dispersion of the active ingredient to be carried out after the spraying (i.e. by final mixing of said active ingredient and of said particles based on said mixture of excipients having been coated with said film) or else before said spraying (i.e. by prior mixing of said active ingredient and of said particles based on said mixture of excipients not yet coated).

As diluent(s) that can be used in said mixture of excipients, use may be made of diluents that are soluble in an aqueous medium of water or saliva type or capable of swelling in this aqueous medium, said diluent(s) being present in said composition according to a total mass fraction ranging from 30% to 90%, and preferably ranging from 50% to 85%.

Said or at least one of said diluent(s) is advantageously:
a water-soluble diluent which is chosen from the group consisting of sugar derivatives, dextrins and maltodextrins, and even more advantageously it is a sugar derivative chosen from the group consisting of lactose, sucrose, trehalose, mannitol, sorbitol, erythritol, maltitol, lactitol and fructose; or else
a diluent capable of swelling in an aqueous medium, which is advantageously chosen from the group consisting of starch derivatives, pregelatinized starches, modified starches (such as cornstarch and potato starch or sodium carboxy-methylstarches), gum derivatives (such as guar gum, xanthan gum or carob gum), derivatives of microcrystalline cellulose (such as sodium carboxymethylcelluloses, calcium carboxymethyl-celluloses, croscarmelloses), and silica derivatives (such as anhydrous colloidal silica).

According to another characteristic of the invention, the mass fraction of said colloidal solution in the dry state in said composition falls within a range of from 0.01% to 10%, preferably of from 0.1% to 5%, and even more preferably of from 0.5% to 3%.

As hydrophilic film-forming compound(s), use is made of a solid or semi-solid which is pulverulent at a temperature of 25° C., which is chosen from the group consisting of carbohydrates, polyethylene glycols, polysaccharides, gum derivatives (such as guar gum, xanthan gum or carob gum), polyglyceride derivatives, and mixtures of several of these compounds.

Advantageously, said hydrophilic film-forming compound according to the invention is a carbohydrate chosen from the group consisting of maltodextrins, dextrins, sorbitol, mannitol and xylitol.

According to a variant of the invention, a polyethylene glycol of weight-average molecular mass Mw ranging from 1000 g/mol to 6000 g/mol is used as hydrophilic film-forming compound.

According to another variant of the invention, a polyglyceride derivative chosen from the group consisting of lauroyl macrogolglycerides or stearoyl macrogolglycerides is used as hydrophilic film-forming compound.

It will be noted that said hydrophilic film-forming compound makes it possible to increase the rate of hydration of the pharmaceutical composition, while at the same time contributing to the cohesion thereof.

As lubricating amphiphilic compound(s), use is advantageously made of a compound chosen from the group consisting of sodium docusate, sodium lauryl ether sulfate, sodium lauryl sulfate, glycerol derivatives (such as glyceryl behenate and glyceryl palmito-stearate), polyethylene glycols, leucine, stearic acid, magnesium stearate, sodium stearyl fumarate and sodium benzoate.

As humectant amphiphilic compound(s), use is made of a compound which is soluble or dispersible in an aqueous medium, which is chosen from the group consisting of lipoamino acids (such as capryloyl glycine and lauroyl proline), sorbitans and their ethoxylated derivatives, lecithin derivatives, polyethylene glycol derivatives, polyglyceride derivatives, poloxamers (such as the poloxamers “188” or “407”), sugars and sugar derivatives (such as sucrose palmitate).

Advantageously, said humectant amphiphilic compound according to the invention is a sorbitan stearate or else a polysorbate by way of ethoxylated sorbitan derivative.

According to a variant of the invention, a polyethylene glycol derivative chosen from the group consisting of cetomacrogols 1000 and macrogol-25 cetostearyl ethers is used as humectant amphiphilic compound.

According to another variant of the invention, a polyglyceride derivative of lauroyl macrogolglyceride or stearoyl macrogolglyceride type, such as a stearoyl macrogol-32 glyceride or a lauroyl macrogol-32 glyceride, is used as humectant amphiphilic compound.

Said mixture of excipients used in the composition according to the invention can also comprise, in combination with said active ingredient(s) and said diluent(s), one or more cohesion agent(s) (often called “gliding agents” by those skilled in the art) such as, in a nonlimiting manner, microcrystalline cellulose or one of its derivatives, a dextrin such as maltodextrin, a lactose, a calcium phosphate derivative or a starch derivative, said cohesion agent(s) being present in said composition according to a mass fraction ranging from 0.1% to 40%.

These cohesion agents are used to facilitate the production of the tablets and to maintain the friability of the latter below 1%. The mass fraction, in said composition according to the invention, of the cohesion agent(s) can be between 0.1% and 40%, and this mass fraction is preferably less than 20%.

The pharmaceutical composition according to the invention can contain variable proportions of active ingredient(s), typically from 0.1% to 50%.

By way of example, and which is in no way limiting, mention may be made of:
antiulcer agents, such as famotidine, omeprazol, lanzoprazol,
antiemetics, such as ondansetron, granisetron, dolasetron, domperidone, metoclopramide,
antihypertensives, such as enalapril, losartan, candesartan, valsartan, lisinopril, ramipril, doxazosin, terazosin,
antihistamines, such as loratadine, cetirizine,
antipsychotics, such as risperidone, olanzapine, quetiapine,
antidepressants, such as paroxetine, fluoxetine, mirtazapine,
analgesics and anti-inflammatories, such as piroxicam,
blood-cholesterol-lowering agents, such as simvastatin, lovastatin, pravastatin,
antimigraine agents, such as zolmitriptan, naratriptan, rizatriptan, sumatriptan,
antiepileptics, such as lamotrigine,
anti-Parkinsonian agents, such as selegiline, apomorphine,
anxiolytics, such as diazepam, lorazepam, zolpidem,
anti-asthmatics, such as zafirlukast, montelukast,
erectile dysfunction agents, such as sildenafil,
antipyretics, such as paracetamol, acetylsalicylic acid, ibuprofen, etc.

It will be noted that all these active ingredients can be used equally in their base form or in a salt, hydrate, solvate and isomer form.

The pharmaceutical composition according to the invention can also contain:
sweeteners, such as sodium saccharinate or aspartame,
flavorings, and/or
lubricants, such as magnesium stearate, stearic acid, sodium stearyl fumarate, talc, a PEG, or pulverulent lipid derivatives such as, for example, glyceryl tribehenate.

A galenic form according to the invention, which gives rapid oral disintegration, comprises an uncoated (i.e. non-film-coated) tablet which consists of said pharmaceutical composition as defined above.

In general, these galenic forms according to the invention can be used for a therapeutic and/or prophylactic treatment of a human or animal organism.

A method for producing, according to the invention, said pharmaceutical composition as defined above comprises the following steps:
a) an oil-in-water or water-in-oil aqueous colloidal solution comprising, firstly, an aqueous phase and, secondly, a self-emulsifying system is prepared;
b) the colloidal solution obtained in a) is sprayed:
   (i) onto solid particles consisting of an active ingredient dispersed in a mixture of excipients comprising at least one diluent, or
   (ii) onto solid particles consisting only of said mixture of excipients (i.e. consisting of a non-medicinal premix),
      in such a way that the (sprayable colloidal solution/particles intended to receive said colloidal solution) mass ratio is less than or equal to 10%;
c) the colloidal solution sprayed in b) is dried so as to obtain:
   in case (i), said pharmaceutical composition consisting of said solid particles comprising said active ingredient, which are coated with a film; or
   in case (ii), an intermediate composition consisting of said solid particles of excipients, which are coated with said film, and then
d) optionally, in this case (ii), said active ingredient is dispersed in said intermediate composition obtained in c), so as to obtain said pharmaceutical composition.

Preferably, it is chosen to incorporate said active ingredient(s) into the pharmaceutical composition subsequent to the spraying, as indicated in step d), in particular in the case where said or at least one of said active ingredient(s) is sensitive to water.

According to another characteristic of this method according to the invention, said self-emulsifying system comprises, in combination, as defined above, said hydrophilic film-forming compound(s), said lubricating amphiphilic compound(s) and said humectant amphiphilic compound(s).

Advantageously, the colloidal solution prepared in step a) comprises a mass fraction of said self-emulsifying system which is less than or equal to 10%, and a mass fraction of water which is greater than or equal to 90%.

Preferably, the colloidal solution obtained in step a) is heated to a temperature ranging from 30° C. to 70° C., for carrying out step b) at such a temperature ranging from 40° C. to 70° C.

In fact, the applicant has discovered that this temperature range advantageously makes it possible to optimize the prehydration of said solid particles through improved wetting and improved hold of the sprayed film of colloidal solution, in comparison with spraying carried out at ambient temperature (for example, 25° C.).

Also preferably, the viscosity of said colloidal solution to be sprayed in step b) falls within a range of from 2 mPa·s to 40 mPa·s at 50° C., and the sprayed droplets have an average size ranging substantially from 10 μm to 100 μm, and preferably ranging from 50 μm to 70 μm. It will be noted that this spraying can in fact be likened to nebulization in the specific case of droplets whose average size varies substantially from 10 to 50 μm.

It will also be noted that this reduced size of the droplets is particularly suitable for obtaining said prehydrating thin film according to the invention which surface-coats said solid particles without causing them to adhere to one another.

According to another characteristic of this method according to the invention, the ratio of the apparent density of the composition obtained in step c) or d) to the apparent density of the solid particles before spraying is between 1 and 1.2.

According to another characteristic of the invention, the pharmaceutical composition thus obtained has, before compression, an apparent density which, measured according to the European Pharmacopoeia by means of a packing volume meter (via 3 measurements of apparent volumes V₀, V₁₀ and V₅₀₀-V₁₀ representative of the packing ability), falls within a range of from 0.4 to 0.9 g/ml.

Furthermore, said particles coated with the film according to the invention exhibit, after spraying and drying, an increase in size which is less than or equal to 20%.

The abovementioned characteristics of the present invention, and also others, will be understood more clearly upon reading the following description of several examples of implementation of the invention, given by way of nonlimiting illustration.

### EXAMPLES

### 1) “Control” Tablet 1 and Tablet 2 According to the Invention of Placebo Type

a) Preparation of Tablets:

“Control” tablets and tablets according to the invention giving rapid disintegration, which are all of placebo type, i.e. devoid of any active ingredient, were prepared.

These “control” placebo tablets and placebo tablets according to the invention consist respectively of pulverulent compositions 1 and 2 having the following formulations in the dry state (as mass fractions in table 1a hereinafter):

TABLE 1a

| | COMPOSITIONS | |
|---|---|---|
| | 1 | 2 |
| Solid particles of excipients: | | |
| PHARMABURST ® (mannitol-based diluent) | 76% | 75.81% |
| Strawberry flavoring | 1% | 1% |
| Aspartame | 1% | 1% |
| Sodium stearyl fumarate | 2% | 2% |
| Microcrystalline cellulose (cohesion | 20% | 20% |
| agent) | | |
| Film of a colloidal solution after drying | | |
| based on water and on the following self- | | |
| emulsifying system: | | |
| maltodextrin (film-forming hydrophilic | 0% | 0.08% |
| compound) | | |
| sodium lauryl sulfate (lubricating | 0% | 0.03% |
| amphiphilic compound) | | |
| “GELUCIRE 44/14” (humectant amphiphilic | 0% | 0.08% |
| compound) | | |

It will be noted that “control” composition 1 was not prehydrated, unlike composition 2 according to the invention, which was prehydrated by spraying, onto the solid particles of excipients, the above-mentioned aqueous colloidal solution, which is of oil-in-water type and which was dried in an oven after spraying.

Table 1b hereinafter gives details of the formulation used to obtain this aqueous colloidal solution (mass fractions):

TABLE 1b

| | |
|---|---|
| Maltodextrin | 1.6% |
| Sodium lauryl sulfate | 0.6% |
| “GELUCIRE 44/14” | 1.6% |
| Water | 96.2% |

This film-forming colloidal solution was heated to 50° C. and then finely sprayed at this same temperature according to a sprayed volume equal to 5 ml, it being specified that its viscosity at this temperature was 3 mPa·s.

Furthermore, the sprayed droplets had an average size ranging substantially from 50 μm to 70 μm.

This composition 2 obtained is such that the particles of excipients are surface-coated with a film of reduced thickness, such that the particles thus coated do not form a granule (i.e. these particles do not adhere to one another despite the spraying, and have, after said spraying, a size substantially identical to their initial size).

Table 2 hereinafter gives the apparent volume and the apparent density of compositions 1 and 2 before compression of said compositions.

TABLE 2

COMPOSITIONS

| | 1 | 2 |
|---|---|---|
| Apparent volume (cm³) | 190 | 186 |
| Apparent density (g/cm³) | 0.526 | 0.537 |

It will be noted that the absence of granulation, which characterizes the spraying carried out in order to obtain composition 2 according to the invention, is reflected by an apparent density of this composition 2, before compression, which is virtually unchanged following the spraying and the drying, i.e. substantially equal to 0.526 g/cm³.

Each of these “control” tablets and tablets according to the invention, of round and flat type, has a diameter of 12 mm, an average thickness of 2.8 mm, a final average mass of 320 mg, an average hardness of 21 Newtons and a friability of the order of 0.45%.

b) In Vitro Tests for Hydration of these Tablets:

Comparative tests for rate of hydration, relating to six “control” tablets 1 and six tablets 2 according to the invention, were carried out. The conditions followed for these tests were the following:
volume V of each tablet: 317 mm³, with V=area of the surface of the flat upper part of the tablet×height of the tablet), and
volume of water deposited on each tablet: 320 mm³.

320 mm³ of water were deposited at the surface of each tablet, and then the time taken for all the water to disappear from the surface of the tablets was measured. The rate of hydration was then calculated, this rate in fact corresponding to the time required for one mm³ of water to be adsorbed by the corresponding tablet.

Table 3 hereinafter gives the average results obtained:

TABLE 3

| | COMPOSITIONS | | | |
|---|---|---|---|---|
| | 1 | | 2 | |
| Absorption time (in seconds) | 33 | s | 20 | s |
| Rate of hydration (per 1 mm³) | 0.102 | s/mm³ | 0.0625 | s/mm³ |

These results show that the tablets 2 according to the invention, which are obtained by spraying said prehydrating colloidal solution onto said particles of excipients without carrying out granulation, adsorb water approximately 1.6 times faster, per 1 mm³, than the “control” tablets 1 which are devoid of such a prehydrating colloidal solution, which reflects a rate of hydration which is clearly improved for these tablets 2 according to the invention.

### 2) Tablets 3 According to the Invention

a) Preparation of Tablets 3:

Pulverulent pharmaceutical compositions 3 according to the invention were prepared in a manner similar to that described for the placebo compositions 2 according to the invention of § 1) above, except that, after the spraying of the aqueous colloidal solution onto a premix of solid particles of excipients, an active ingredient was dispersed in the particles of excipients coated with the spray film. Each composition 3 has the following formulation in the dry state (as mass fractions in table 4a hereinafter):

TABLE 4a

| | COMPOSITION |
|---|---|
| | 3 |
| Premix of particles of excipients coated by | |
| spraying: | |
| PHARMABURST ® (mannitol-based diluent) | 78% |
| Mint flavoring | 1% |
| Aspartame | 1% |
| “PRUV” (antiadhesive lubricant for processing) | 4% |
| “AVICEL Ph 200” microcrystalline cellulose | 10% |
| (cohesion agent) | |
| Film of a colloidal solution according to the | |
| invention after drying | |
| based on water and on the following self- | |
| emulsifying system: | |
| “PEG 4000” (film-forming hydrophilic compound) | 2% |
| sodium lauryl ether sulfate (lubricating | 0.5% |
| amphiphilic compound) | |
| “GELUCIRE 44/14” (humectant amphiphilic | 0.5% |
| compound) | |
| Glycerol | 2% |
| Risperidone (active ingredient) | 1% |

Table 4b hereinafter gives details of the formulation used to obtain this aqueous colloidal solution (mass fractions):

TABLE 4b

| | |
|---|---|
| “PEG 4000” | 20% |
| Sodium lauryl ether sulfate | 5% |
| “GELUCIRE 44/14” | 5% |
| Glycerol | 20% |
| Water | 50% |

This film-forming colloidal solution was heated to 50° C. and then sprayed, in fine droplets, onto moving particles of excipients at this same temperature, according to a sprayed volume equal to 10 ml.

These fine droplets were produced via a conventional spray nozzle of hollow cone type. Tests showed that the period necessary and sufficient for spraying these 10 ml of colloidal solution onto the particles of excipients was 3 minutes.

The operating protocol followed was the following:
the premix of particles of excipients to be coated (this premix being devoid of active ingredient) was agitated via a jacketed or non-jacketed planetary mixer; then
the film-forming colloidal solution was sprayed onto this moving excipient premix, for 3 minutes; then
the residual moisture of the particles thus coated was measured, and was of the order of 7%; then
the particles of the premix coated with this colloidal solution were dried, following the spraying, for 5 hours in an incubator at a temperature of 50° C.; then
the residual moisture of the coated and dried particles was measured, and was of the order of 2.6%; then
the active ingredient was dispersed by mixing, while lubricating the whole.

It will be noted that the absence of granulation, which characterizes the spraying carried out so as to obtain composition 3, is reflected by an apparent density of this composition 3, before compression, which is unchanged following the spraying and the drying.

It will also be noted that virtually the same protocol could be used to obtain a composition in which the spraying of the colloidal solution was directly carried out onto the solid particles incorporating the active ingredient in addition to the excipients.

After compression, tablets 3 according to the invention giving rapid oral disintegration, which each contain 1 mg of risperidone as active ingredient, were obtained.

Each tablet 3 has a final mass of 100 mg, an average hardness of 21 Newtons, a thickness of 2.2 mm and a friability of the order of 0.45%.

b) In Vivo Tests for Hydration of these Tablets 3:

Hydration tests were carried out on six human individuals. The rate of hydration of these tablets 3 according to the invention, defined here as being the time required to obtain oral disintegration or crumbling of these tablets through the action of saliva, was determined by preparing an average over the six individuals. A rate of hydration of 25 seconds was thus obtained for these tablets 3.

### 3) Tablets 4 According to the Invention

a) Preparation of Tablets 4:

Pulverulent pharmaceutical compositions 4 according to the invention were prepared in a manner similar to that described in § 2) above (i.e. after spraying the aqueous colloidal solution onto the solid particles of excipients, an active ingredient was dispersed in the particles of excipients coated with the spray film). Each composition 4 has the following formulation in the dry state (as mass fractions in table 5 hereinafter):

TABLE 5

COMPOSITION
4

| | |
|---|---|
| Premix of particles of excipients coated by | |
| spraying: | |
| Mannitol (diluent) | 81.4% |
| Mint flavoring | 1% |
| Aspartame | 1% |
| “PRUV” (antiadhesive lubricant for processing) | 4% |
| “AVICEL Ph 200” microcrystalline cellulose | 10% |
| (cohesion agent) | |
| Film of a colloidal solution according to the | |
| invention after drying | |
| based on water and on the following self- | |
| emulsifying system: | |
| “PEG 4000” (film-forming hydrophilic compound) | 0.1% |
| “PEG 400” (film-forming hydrophilic compound) | 0.4% |
| Dietary lecithin (lubricating amphiphilic | 1% |
| compound) | |
| “GELUCIRE 44/14” (humectant amphiphilic | 0.5% |
| compound) | |
| Risperidone (active ingredient) | 0.6% |

It will be noted that the absence of granulation, which characterizes the spraying carried out to obtain composition 4, is reflected by an apparent density of this composition 4, before compression, which is unchanged following the spraying and the drying.

After compression, tablets 4 according to the invention giving rapid oral disintegration, which each contain 1 mg of risperidone as active ingredient, were obtained.

Each tablet 4 has a final mass of 166 mg, an average hardness of 22 Newtons, a thickness of 2.08 mm and a friability of the order of 0.75%.

b) In vivo tests for hydration of these tablets 4:

Hydration tests were carried out on six human individuals, by implementing the process as described in § 2) above. A rate of hydration of 18 seconds was thus obtained for these tablets 4.

### 4) Tablets 5 According to the Invention

a) Preparation of Tablets 5:

Pulverulent pharmaceutical compositions 5 according to the invention were prepared in a manner similar to that described in § 2) above. Each composition 5 has the following formulation in the dry state (as mass fractions in table 6 hereinafter):

TABLE 6

COMPOSITION
5

| | |
|---|---|
| Premix of particles of excipients coated by | |
| spraying: | |
| PHARMABURST ® (mannitol-based diluent) | 67.87% |
| Mint flavoring | 1% |
| Aspartame | 1% |
| “PRUV” (antiadhesive lubricant for processing) | 2% |
| “AVICEL Ph 200” microcrystalline cellulose | 10% |
| (cohesion agent) | |
| Film of an aqueous microemulsion according to the | |
| invention after drying | |
| based on water and on the following self- | |
| emulsifying system: | |
| “GELUCIRE 44/14” (film-forming hydrophilic | 8% |
| compound) | |
| Magnesium stearate (lubricating amphiphilic | 2% |
| compound) | |
| Sodium lauryl sulfate (humectant amphiphilic | 2% |
| compound) | |
| Ordansetron (active ingredient) | 6.13% |

It will be noted that the absence of granulation, which characterizes the spraying carried out to obtain composition 5, is reflected by an apparent density of this composition 5, before compression, which is unchanged following the spraying and the drying.

After compression, tablets 5 according to the invention giving rapid oral disintegration, which each contain 4 mg of ordansetron as active ingredient, were obtained.

Each tablet 5 has a final mass of 65 mg, an average hardness of 19 Newtons, a thickness of 2.08 mm and a friability of the order of 0.58%.

b) In Vivo Tests for Hydration of these Tablets 5:

Hydration tests were carried out on six human individuals, by implementing the process as described in § 2) above. A rate of hydration of 22 seconds was thus obtained for these tablets 5.

### 5) Tablets 6 According to the Invention

a) Preparation of Tablets 6:

Pulverulent pharmaceutical compositions 6 according to the invention were prepared in a manner similar to that described in § 2) above. Each composition 6 has the following formulation in the dry state (as mass fractions in table 7 hereinafter):

TABLE 7

COMPOSITION
6

| | |
|---|---|
| Premix of particles of excipients coated by | |
| spraying: | |
| Erythritol (diluent) | 59.87% |
| Strawberry flavoring | 1.5% |
| Aspartame | 1% |
| “PRUV” (antiadhesive lubricant for processing) | 2% |
| “AVICEL Ph 200” microcrystalline cellulose | 20% |
| (cohesion agent) | |
| Film of an aqueous microemulsion according to the | |
| invention after drying | |
| based on water and on the following self- | |
| emulsifying system: | |
| “PEG 1500” (film-forming hydrophilic compound) | 1% |
| “GELUCIRE 50/13” (humectant amphiphilic | 5.5% |
| compound) | |
| Magnesium stearate (lubricating amphiphilic | 2% |
| compound) | |
| Sodium lauryl sulfate (lubricating amphiphilic | 1% |
| compound) | |
| Loperamide (active ingredient) | 6.13% |

It will be noted that the absence of granulation, which characterizes the spraying carried out to obtain composition 6, is reflected by an apparent density of this composition 6, before compression, which is unchanged following the spraying and the drying.

After compression, tablets 6 according to the invention giving rapid oral disintegration, which each contain 2 mg of loperamide as active ingredient, were obtained.

Each tablet 6 has a final mass of 166 mg, an average hardness of 22 Newtons, a thickness of 2.08 mm and a friability of the order of 0.75%.

b) In Vivo Tests for Hydration of these Tablets 6:

Hydration tests were carried out on six human individuals, by implementing the process described in § 2) above. A rate of hydration of 19 seconds was thus obtained for these tablets 6.

### 6) Tablets 7 According to the Invention

a) Preparation of Tablets 7:

Pulverulent pharmaceutical compositions 7 according to the invention were prepared in a manner similar to that described in § 2) above. Each composition 7 has the following formulation in the dry state (as mass fractions in table 8 hereinafter):

TABLE 8

COMPOSITION
7

| | |
|---|---|
| Premix of particles of excipients coated by | |
| spraying: | |
| PHARMABURST ® (mannitol-based diluent) | 50.17% |
| Mint flavoring | 1% |
| Aspartame | 2% |
| “PRUV” (antiadhesive lubricant for processing) | 2% |
| “AVICEL Ph 200” microcrystalline cellulose | 10% |
| (cohesion agent) | |
| Film of an aqueous microemulsion according to the | |
| invention after drying | |
| based on water and on the following self- | |
| emulsifying system: | |
| “PEG 1000” (film-forming hydrophilic compound) | 0.8% |
| Sodium lauryl sulfate (humectant amphiphilic | 0.5% |
| compound) | |
| Glyceryl tribehenate (lubricating amphiphilic | 0.2% |
| compound) | |
| Paracetamol (active ingredient) | 33.33% |

It will be noted that the absence of granulation, which characterizes the spraying carried out to obtain composition 7, is reflected by an apparent density of this composition 7, before compression, which is unchanged following the spraying and the drying.

After compression, tablets 7 according to the invention giving rapid oral disintegration, which each contain 500 mg of paracetamol as active ingredient, were obtained.

Each tablet 7 has a final mass of 1500 mg, an average hardness of 25 Newtons, a thickness of 3.5 mm and a friability of the order of 0.75%.

b) In Vivo Tests for Hydration of these Tablets 7:

Hydration tests were carried out on six human individuals, by implementing the process as described in § 2) above. A rate of hydration of 30 seconds was thus obtained for these tablets 7.

### 7) Tablets 8 According to the Invention

a) Preparation of Tablets 8:

Pulverulent pharmaceutical compositions 8 according to the invention were prepared in a manner similar to that described in § 2) above. Each composition 8 has the following formulation in the dry state (as mass fractions in table 9 hereinafter):

TABLE 9

| COMPOSITION | 8 |
|---|---|
| Premix of particles of excipients coated by | |
| spraying: | |
| Mannitol (diluent) | 61.65% |
| Mint flavoring | 1% |
| Aspartame | 2% |
| “PRUV” (antiadhesive lubricant for processing) | 2% |
| “AVICEL Ph 200” microcrystalline cellulose | 10% |
| (cohesion agent) | |
| “PEG 1000” | 0.3% |
| Magnesium stearate | 2% |
| Film of a colloidal solution according to the | |
| invention after drying | |
| based on water and on the following self- | |
| emulsifying system: | |
| “Poloxamer 188” (film-forming hydrophilic | 0.4% |
| compound) | |
| Sodium lauryl sulfate (lubricating amphiphilic | 0.15% |
| compound) | |
| “GELUCIRE 44/14” (humectant amphiphilic compound) | 0.5% |
| Paracetamol (active ingredient) | 20% |

It will be noted that the absence of granulation, which characterizes the spraying carried out to obtain composition 8, is reflected by an apparent density of this composition 8, before compression, which is unchanged following the spraying and the drying.

After compression, tablets 8 according to the invention giving rapid oral disintegration, which each contain 500 mg of paracetamol as active ingredient, were obtained.

Each tablet 8 has a final mass of 2500 mg, an average hardness of 29 Newtons, a thickness of 3 mm and a friability of the order of 0.75%.

b) In Vivo Tests for Hydration of these Tablets 8:

Hydration tests were carried out on six human individuals, by implementing the process as described in § 2) above. A rate of hydration of 28 seconds was thus obtained for these tablets 8.

### 8) Tablets 9 According to the Invention

a) Preparation of Tablets 9:

Pulverulent pharmaceutical compositions 9 according to the invention were prepared in a manner similar to that described in § 2) above. Each composition 9 has the following formulation in the dry state (as mass fractions in table 10 hereinafter):

TABLE 10

| COMPOSITION | 9 |
|---|---|
| Premix of particles of excipients coated by | |
| spraying: | |
| PHARMABURST ® (mannitol-based diluent) | 79.25% |
| Mint flavoring | 1% |
| Aspartame | 2% |
| “PRUV” (antiadhesive lubricant for processing) | 4% |
| “AVICEL Ph 200” microcrystalline cellulose | 10% |
| (cohesion agent) | |
| Film of an aqueous microemulsion according to the | |
| invention after drying | |
| based on water and on the following self- | |
| emulsifying system: | |
| “PEG 1500” (film-forming hydrophilic compound) | 0.5% |
| “Poloxamer 188” (humectant amphiphilic compound) | 0.5% |
| “PRECIROL ATO 5” (lubricating amphiphilic | 0.25% |
| compound) | |
| Zolmitriptan (active ingredient) | 2.5% |

It will be noted that the absence of granulation, which characterizes this spraying, is reflected by an apparent density of this composition 9, before compression, which is unchanged following the spraying and the drying.

After compression, tablets 9 according to the invention giving rapid oral disintegration, which each contain 2.5 mg of zolmitriptan as active ingredient, were obtained. Each tablet 9 has a final mass of 100 mg, an average hardness of 21 Newtons, a thickness of 2.07 mm and a friability of the order of 0.7%.

b) In Vivo Tests for Hydration of these Tablets 9:

Hydration tests were carried out on six human individuals, by implementing the process as described in § 2) above. A rate of hydration of 28 seconds was thus obtained for these tablets 9.

### 9) Tablets 10 According to the Invention

a) Preparation of Tablets 10:

Pulverulent pharmaceutical compositions 10 according to the invention were prepared in a manner similar to that described in § 2) above. Each composition 10 has the following formulation in the dry state (as mass fractions in table 11 hereinafter):

TABLE 11

| COMPOSITION | 10 |
|---|---|
| Premix of particles of excipients coated by | |
| spraying: | |
| Mannitol (diluent) | 32.77% |
| Sorbitol (diluent) | 15% |
| Mint flavoring | 1% |
| Aspartame | 2% |
| “AVICEL Ph 200” microcrystalline cellulose | 10% |
| (cohesion agent) | |
| Film of an aqueous microemulsion according to the | |
| invention after drying | |
| based on water and on the following self- | |
| emulsifying system: | |
| “PEG 1500” (film-forming hydrophilic compound) | 3% |
| “Poloxamer 188” (humectant amphiphilic compound) | 0.6% |
| “Magnesium stearate” (lubricating amphiphilic | 2% |
| compound) | |
| “GELUCIRE 44/14” (humectant amphiphilic compound) | 0.3% |
| Ibuprofen (active ingredient) | 33.33% |

It will be noted that the absence of granulation, which characterizes this spraying, is reflected by an apparent density of this composition 10, before compression, which is unchanged following the spraying and the drying.

After compression, tablets 10 according to the invention giving rapid oral disintegration, which each contain 200 mg of ibuprofen as active ingredient, were obtained. Each tablet 10 has a final mass of 600 mg, an average hardness of 21 Newtons, a thickness of 2.07 mm and a friability of the order of 0.7%.

b) In Vivo Tests for Hydration of these Tablets 10:

Hydration tests were carried out on six human individuals, by implementing the process as described in § 2) above. A rate of hydration of 28 seconds was thus obtained for these tablets 10.

1. A pulverulent pharmaceutical composition which can be used for constituting, after compression, a galenic form giving rapid oral disintegration, said composition comprising solid particles based on a mixture of excipients which comprises at least one diluent, said particles being coated with a film based on an aqueous colloidal solution which is provided for prehydrating said composition, such that said particles thus coated are free from one another in said composition, characterized in that said colloidal solution comprises a self-emulsifying system comprising, in combination:
at least one hydrophilic film-forming compound,
at least one lubricating amphiphilic compound, and
at least one humectant amphiphilic compound.

2. The pharmaceutical composition as claimed in claim 1, characterized in that the mass fraction of said colloidal solution in the dry state in said composition falls within a range of from 0.01% to 10%.

3. The pharmaceutical composition as claimed in claim 2, characterized in that the mass fraction of said colloidal solution in the dry state in said composition falls within a range of from 0.1% to 5%.

4. The pharmaceutical composition as claimed in claim 3, characterized in that the mass fraction of said colloidal solution in the dry state in said composition falls within a range of from 0.5% to 3%.

5. The pharmaceutical composition as claimed claim 1, characterized in that said hydrophilic film-forming compound is a solid which is pulverulent at a temperature of 25° C., which is chosen from the group consisting of carbohydrates, polyethylene glycols, polysaccharides, gum derivatives, polyglyceride derivatives, and mixtures of several of these compounds.

6. The pharmaceutical composition as claimed in claim 5, characterized in that said hydrophilic film-forming compound is a carbohydrate chosen from the group consisting of maltodextrins, dextrins, sorbitol, mannitol and xylitol.

7. The pharmaceutical composition as claimed in claim 5, characterized in that said hydrophilic film-forming compound is a polyethylene glycol of weight-average molecular mass Mw ranging from 1000 g/mol to 6000 g/mol.

8. The pharmaceutical composition as claimed in claim 5, characterized in that said hydrophilic film-forming compound is a polyglyceride derivative chosen from the group consisting of lauroyl macrogolglycerides or stearoyl macrogolglycerides.

9. The pharmaceutical composition as claimed in claim 1, characterized in that said lubricating amphiphilic compound is chosen from the group consisting of sodium docusate, sodium lauryl ether sulfate, sodium lauryl sulfate, glycerol derivatives, polyethylene glycols, leucine, stearic acid, magnesium stearate, sodium stearyl fumarate and sodium benzoate.

10. The pharmaceutical composition as claimed in claim 1, characterized in that said humectant amphiphilic compound is chosen from the group consisting of lipoamino acids, sorbitans and their ethoxylated derivatives, lecithin derivatives, polyethylene glycol derivatives, polyglyceride derivatives, poloxamers, sugars and sugar derivatives.

11. The pharmaceutical composition as claimed in claim 10, characterized in that said humectant amphiphilic compound is a poloxamer.

12. The pharmaceutical composition as claimed in claim 10, characterized in that said humectant amphiphilic compound is a polyethylene glycol derivative chosen from the group consisting of cetomacrogols 1000 and macrogol-25 cetostearyl ethers.

13. The pharmaceutical composition as claimed in claim 10, characterized in that said humectant amphiphilic compound is a polyglyceride derivative of lauroyl macrogolglyceride or stearoyl macrogolglyceride type.

14. The pharmaceutical composition as claimed in claim 1, characterized in that said or each diluent is soluble in an aqueous medium of water or saliva type or is capable of swelling in said aqueous medium, said diluent(s) being present in said composition according to a total mass fraction ranging from 30% to 90%.

15. The pharmaceutical composition as claimed in claim 14, characterized in that said or at least one of said diluent(s) is a water-soluble diluent which is chosen from the group consisting of sugar derivatives, dextrins and maltodextrins.

16. The pharmaceutical composition as claimed in claim 15, characterized in that said or at least one of said diluent(s) comprises a sugar derivative chosen from the group consisting of lactose, sucrose, trehalose, mannitol, sorbitol, erythritol, maltitol, lactitol and fructose.

17. The pharmaceutical composition as claimed in claim 14, characterized in that said or at least one of said diluent(s) is a diluent capable of swelling in an aqueous medium, which is chosen from the group consisting of starch derivatives, pregelatinized starches, modified starches, gum derivatives, derivatives of microcrystalline cellulose, and silica derivatives.

18. The pharmaceutical composition as claimed in claim 1, characterized in that said mixture of excipients also comprises at least one cohesion agent, such as microcrystalline cellulose or one of its derivatives, a dextrin, a lactose, a calcium phosphate derivative or a starch derivative, said cohesion agent(s) being present in said composition according to a mass fraction ranging from 0.1% to 40%.

19. The pharmaceutical composition as claimed in claim 1, characterized in that it has, before compression, an apparent density, measured according to the European Pharmacopoeia, which falls within a range of from 0.4 to 0.9 g/ml.

20. The pharmaceutical composition as claimed in claim 1, characterized in that it comprises at least one active ingredient in the dispersed state in said mixture of excipients.

21. A galenic form giving rapid oral disintegration and comprising an uncoated tablet, characterized in that said tablet consists of a pharmaceutical composition as claimed in claim 1.

22. The galenic form as claimed in claim 21, characterized in that said tablet has a friability, measured in accordance with the European Pharmacopoeia, which is less than 1%.

23. The galenic form as claimed in claim 21, characterized in that said tablet has a breaking strength, measured in accordance with the European Pharmacopoeia, which is greater than 15 Newtons.

24. A method for producing a pharmaceutical composition comprising the following steps:
a) an aqueous colloidal solution of oil-in-water or water-in-oil type comprising, firstly, an aqueous phase and, secondly, a self-emulsifying system is prepared;
b) the colloidal solution obtained in a) is sprayed:
(i) onto solid particles consisting of an active ingredient dispersed in a mixture of excipients comprising at least one diluent, or
(ii) onto solid particles consisting only of said mixture of excipients,
in such a way that the (sprayable colloidal solution/particles intended to receive said colloidal solution) mass ratio is less than or equal to 10%;
c) the colloidal solution sprayed in b) is dried so as to obtain:
in case (i), said pharmaceutical composition consisting of said solid particles comprising said active ingredient, which are coated with a film; or
in case (ii), an intermediate composition consisting of said solid particles of excipients, which are coated with said film, and then
d) optionally, in this case (ii), said active ingredient is dispersed in said intermediate composition obtained in c), so as to obtain said pharmaceutical composition.

25. The method as claimed in claim 24, characterized in that said self-emulsifying system comprises, in combination:
at least one hydrophilic film-forming compound, such as a solid which is pulverulent at a temperature of 25° C., which is chosen from the group consisting of carbohydrates, polyethylene glycols, polyglyceride derivatives, and mixtures of several of these compounds,
at least one lubricating amphiphilic compound, such as a compound chosen from the group consisting of sodium docusate, sodium lauryl ether sulfate, sodium lauryl sulfate, glycerol derivatives, polyethylene glycols, leucine, stearic acid, magnesium stearate, sodium stearyl fumarate and sodium benzoate, and
at least one humectant amphiphilic compound, such as a compound chosen from the group consisting of lipoamino acids, sorbitans and their ethoxylated derivatives, lecithin derivatives, polyethylene glycol derivatives, polyglyceride derivatives, poloxamers, sugars and sugar derivatives.

26. The method as claimed in claim 24, characterized in that said colloidal solution prepared in step a) comprises:
a mass fraction of said self-emulsifying system of less than or equal to 10%, and
a mass fraction of water greater than or equal to 90%.

27. The method as claimed in claim 24, characterized in that step b) is carried out at a temperature ranging from 40° C. to 70° C.

28. The method as claimed in claim 24, characterized in that the viscosity at 50° C. of said colloidal solution to be sprayed in step b) falls within a range of from 2 mPa·s to 40 mPa·s.

29. The method as claimed in claim 24, characterized in that the ratio of the apparent density of the composition obtained in step c) or d) to the apparent density of the solid particles before spraying is between 1 and 1.2.
